(19) United States
(12) Patent Application Publication
(10) Pub. No.: US 2015/0297062 A1
(43) Pub. Date: Oct. 22, 2015

(54) INTEGRATED ENDOSCOPE

(71) Applicant: GOLENBERG Lavie, (US)

(72) Inventors: Lavie Golenberg, Farmington Hills, MI (US); Prem Sivakumar, Detroit, MI (US)

(21) Appl. No.: 14/411,877

(22) PCT Filed: Jun. 28, 2013
(86) PCT No.: PCT/US2013/048515

§ 371 Date: Dec. 29, 2014

Related U.S. Application Data
(60) Provisional application No. 61/665,877, filed on Jun. 28, 2012.

(30) Foreign Application Priority Data
Jun. 28, 2012 (US) 61665877

(51) Int. Cl. A61B 1/00; H04N 5/272; A61B 1/045; H04N 5/38

(52) U.S. Cl. CPC A61B 1/00108; H04N 5/38; H04N 5/272; A61B 1/00006; A61B 1/00009; A61B 1/00018; A61B 1/00048; A61B 1/045

(57) ABSTRACT

An endoscope that integrates the functions of an optical tower into a portable device, while eliminating the use of cords or cables that carry light, video signals or images, and power to the endoscope that may interfere with the movement of a surgeon and the members of the surgical team, or other operators in non-medical related applications is provided. The endoscope incorporates a camera, an image processor, a light source, a transmitter, a communication interface, a control interface, and one or more of a power source in a single portable unit or enclosure. The camera is in electrical communication with the image processor and supplies images and video to the image processor obtained via an elongated endoscope tube. The light source illuminates a viewing field of the endoscope via the elongated tube.

### CROSS-REFERENCE TO RELATED APPLICATION

This application claims priority of U.S. Provisional Patent Application Ser. No. 61/665,877 filed Jun. 28, 2012, which is incorporated herein by reference.

### FIELD OF THE INVENTION

The present invention in general relates to medical and inspection devices and in particular to a wireless portable endoscope with integrated power, lighting, and video processor.

### BACKGROUND OF THE INVENTION

An endoscope is a device with a light attached to one end, used to look inside or inspect a region inside a confined area or a specific body cavity or organ. Endoscopes are inserted through a natural opening, such as the mouth or rectum and are commonly used to detect ulcers, inflammation, erosions, polyps, strictures, malignancies, varices, and bleeding sites. In a surgical procedure, an endoscope is inserted through a small incision that permits minimally invasive procedures that improve patient care and minimize recovery time. Any medical procedure that uses endoscope equipment is called an endoscopy. Particular medical procedures that employ endoscopes include arthroscopy (orthopedic joints), bronchoscopy (lung), colonoscopy (colon), cystoscopy (bladder), gastroscopy (upper gastrointestinal tract), laryngoscopy (larynx), laparoscopy (abdomen, peritoneal cavity, ovaries, fallopian tubes and uterus), nephroscopy (kidney), otoscopy (ear), and rhinoscopy (nose).

Endoscopes capture images through a long tube, which can be rigid or flexible. Images may be captured by a purely fiber optic scope with a bundle of glass fibers that collect the lighted images at one end and transfer them to an eye piece, or video images may be obtained using a small, optically sensitive computer chip at the end of the scope. The computer chip transmits electronic signals up the scope to a computer which then displays the image on a large video screen. Advances in lighting technologies, such as light emitting diodes (LED) have improved the imaging performance of endoscopes. Additional instruments for cutting, grasping and other functions are often attached to the endoscope, or are supplied via an open channel in the endoscope to allow other instruments to pass through in order to perform biopsies, remove polyps or inject solutions, as needed. Endoscopes are also well suited for a number of industrial applications such inspections and are synonymously referred to herein as borescopes.

While endoscopes offer many advantages to physicians and patients, the use of current endoscopes require the use of an optical tower. Optical towers include a power source for the endoscope, a monitor/display, a light source, and a video receiver and processor. Optical towers are generally quite large, and take up valuable floor space in an operating room. In addition, the use of cords or cables that carry light, video signals or images, and power to the endoscope interfere with the movement of the surgeon and the members of the surgical team.

Thus, there exists a need for an endoscope that integrates the functions of an optical tower into a portable device, while eliminating the use of cords or cables that carry light, video signals or images, and power to the endoscope that may interfere with the movement of a surgeon and the members of the surgical team.

### SUMMARY OF THE INVENTION

An endoscope that integrates the functions of an optical tower into a portable device, while eliminating the use of cords or cables that carry light, video signals or images, and power to the endoscope that may interfere with the movement of a surgeon and the members of the surgical team, or other operators in non-medical related applications is provided. Embodiments of the inventive endoscope incorporate a camera, an image processor, a light source, a transmitter, a communication interface, a control interface, and a power source in a single portable unit or enclosure. The camera is in electrical communication with the image processor and supplies images and video to the image processor obtained via an elongated endoscope tube. The light source illuminates a viewing field of the endoscope via the elongated tube. One or more power sources supply power to the camera, the image processor, the light source, and the transmitter.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates a block diagram of an embodiment of the endoscope in communication with various user and network devices;

FIG. 2 is a schematic diagram illustrating an overall view of communication devices, computing devices, and mediums for interfacing with the inventive endoscope.

### DETAILED DESCRIPTION OF THE PREFERRED EMBODIMENTS

The present invention has utility as an endoscope that integrates the functions of an optical tower into a portable device, while eliminating the use of cords or cables that carry light, video signals or images, and power to the endoscope that may interfere with the movement of a surgeon and the members of the surgical team, or other operators in non-medical related applications. Embodiments of the inventive endoscope incorporate a camera, an image processing unit, a light source, a transmitter, a communication interface, a control interface, and a power source in a single portable unit or enclosure.

Embodiments of the inventive endoscope eliminate the need for an optical tower that traditionally provides a power source, monitor, light source, and video processing. The inventive endoscope is a compact and inexpensive tool for performing endoscopic surgery by eliminating the optical tower currently used for endoscopic surgery. This medical hardware invention saves space through the elimination of the optical tower, and as a result provides surgeons with the flexibility and option to perform endoscopic based surgery in a smaller operating room of a clinic or office setting. It is appreciated that the endoscope is well suited for field hospital usage. The inventive endoscope affords portability, since an optical tower is not necessary for the operation of embodiments of the endoscope, and endoscopic surgery can now be performed anywhere that has carbon dioxide gas, a power source for cauterization, and anesthesia. The inventive endoscope has potential uses for the military and non-governmental agencies that provide medical care in a mobile facility with limited facilities. In addition, embodiments of the invention can also be used in smaller or rural facilities that cannot designate a room purely for endoscopic surgery. Furthermore, the portability of the inventive endoscope allows for non-medical related applications such as industrial inspection, pest control, and remediation, etc.

In certain inventive embodiments, images and video provided by the inventive endoscope are transmitted simultaneously either wirelessly or via a wired connection to personal wearable viewers equipped with heads up display (HUD), the Internet via a router, local area networks (LAN), and other configurable or available forms of communication. In other inventive embodiments, the endoscopic imagery is also readily viewed on a television (TV) screen if broadcasted on a specific frequency. In some inventive embodiments, a user selects transmission frequencies and video channels for the endoscope to account for interfering signals. In addition, a user may be able to switch video channels to access additional informational content or operating room views. A TV adapter is included in some inventive embodiments to provide direct transmission. The ability to send images over networks is beneficial to surgeons not in the operating room that are needed for consultations, educational purposes, as well as remote viewers for non-medical applications. The innovation provided by embodiments of the invention allows a surgeon to keep a headset, video monitor, or remote viewing device in their office, home or other remote location, allowing for quick feedback rather than have the surgeon physically travel to the operating room to provide surgical consultation.

In other inventive embodiments, a user has the option of providing selectable transmission frequencies and video channels for the endoscope to account for interfering signals. In addition, a user may be able to switch video channels to access additional informational content or operating room views. In still other inventive embodiments, the capability to overlay images onto the endoscope's video feed from another source or piece of medical equipment or monitoring device is provided. Overlaid images illustratively include patient vital sign information, augmented reality images, scans from computed tomography (CT), ultrasound images, Doppler flow scans, X-ray, magnetic resonance imaging (MRI), or combinations thereof.

With embodiments of the inventive endoscope, the insertion of and design of surgical tools will remain the same. Trocars are still required to provide the seal for the hand tools and endoscope, and the hand tools are powered for cauterization. While a surgeon manipulates the hand tools, and another surgeon often navigates the endoscope. The electronics (camera and imager, transmitter, lighting, communication interface, power supply) are mounted onto the endoscope in the same manner that the endoscope video camera was mounted onto existing endoscope designs. A light source, including light emitting diodes (LED), is attached to the inventive endoscope by a fiber-optic cable. In certain inventive embodiments, a visual record of the viewing field of the endoscope is collected, and transmit the signal wired or wirelessly to personal video viewers worn by the master surgeon manipulating the hand tools and by the surgeon navigating the endoscope. It is appreciated that in specific inventive embodiments, it is beneficial to have the capability to transmit endoscopic imagery to a TV, especially from remote locations with limited communications infrastructure. This added flexibility would still not require the optical tower common to conventional systems and provides and optional backup in cases where the HUD is not functioning. Additional wired/wireless signals can be transmitted to additional surgeons, nurses, students, and observers as necessary. In non-medical applications, the video features of the inventive endoscope may be used for inspection of remote or hard to reach areas that include, for example, industrial inspection, pest control, remediation such as in pipes, sewers, machinery enclosures, and nuclear facilities. Military and public safety agencies may use the video features for reconnaissance and rescue missions. The user's ability to switch video channels allows a soldier or emergency personnel to see via shared views what their teammates see from their own endoscopes, whether their teammate is alive or dead, providing a higher level of situational awareness.

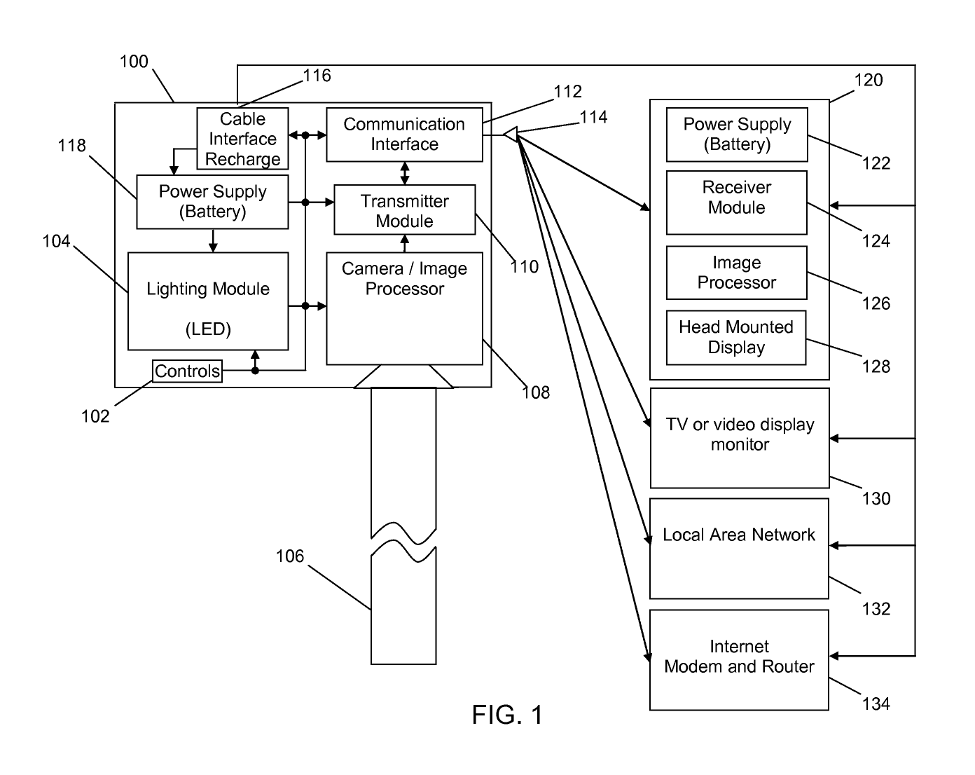

With reference to the attached figures, an inventive endoscope is depicted generally at 100 in FIG. 1. The endoscope 100 includes within an integrated enclosure with optical tower function so as to eliminate the need of an optical tower. Controls 102 are present in the endoscope 100 in electrical communication with lighting module 104, camera/image processor 108, transmitter module 110, and communication interface 112. Controls 102 provide the user with the ability to control the intensity of the lighting with the lighting module 104, camera and imaging parameters of the camera/image processor 108, as well as communication parameters of the transmitter module 110 and communication interface 112. The lighting module 104 provides light via elongated tube 106 to the surgical area of interest with controlled intensity and apertures. Camera and image parameters illustratively include depth of field, frame rate, illumination wavelengths, focus, pixel density, false color, frame size (x/y ratio), noise filtering, and baud rate. The elongated tube 106 can be rigid or flexible, as are conventional to the art. In certain inventive embodiments, images may be captured by a purely fiber optic scope with a bundle of glass fibers that collect the lighted images at one end of the elongated tube 106 and transfer them to an eye piece, or video images may be obtained using a small, optically sensitive computer chip, such as a charged coupled device (CCD) within the camera/image processor 108 at the end of the scope tube 106.

The scope tube 106 besides providing a conduit for collecting images and a pathway for insertion of surgical tools is provided in certain inventive embodiments with a channel for providing suction to an examined area to remove obstructions such as smoke or liquids, or to remove materials such as masses of tissue that are being excised or debulked in a surgical procedure. In addition, the scope tube 106 is provided with a channel to introduce a fluid to a remote tissue area for example a liquid active agent, such as a curable resin, irrigation fluid, air, is provided via the channel to the tissue. Furthermore, other operations that may be conducted via scope tube 106 illustratively include the insertion of a spectroscopy system or the introduction of a manipulator or thermal tools such as an induction heating coil, welding gases (in an industrial pipe testing/repair setting), or cautery tool.

The transmitter module 110 broadcasts the images and video obtained from the camera/image processor 108 via predefined frequencies and protocols including WiFi (802.11 a,b,g,n), WiMax, Bluetooth, near field, cellular protocols, and other existing and contemplated communication protocols. In certain inventive embodiments of the transmitter module 110 may also allow for user selectable transmission frequencies and video channels for the endoscope to account for interfering signals. In addition, a user may be able to switch video channels to access additional informational content or operating room views. In other embodiments, the transmitter module 110 provides the capability to overlay images from another source or piece of medical equipment or monitoring device onto the endoscope video feed via a wired or wireless connection to the overlay source.

In another inventive embodiment, the transmission from the endoscope is encrypted for instances where secure communications are required. The communication interface 112 routes the broadcast signals to an antenna 114, or takes the camera and image signals and routes them to a cable interface 116 for a wired connection via for example composite video, s-video, universal serial bus (USB), high definition media interface (HDMI), digital video interface (DVI), coax cable, or other wired standards. Power supply 118 may be directly connected to a 110-240 V AC electrical outlet, or may be a battery that is or is not rechargeable. A rechargeable battery may be charged while connected to an outlet, or via a communication cable such as a USB cable. Power supply 118 may act as a power source or supply to peripheral devices, such as a vacuum for suction or other medical assist devices, via a USB connector or DC power receptacle. As noted above, embodiments of the inventive endoscope are portable self-contained units that are configured for wired or wireless operation thereby eliminating the need for an optical tower.

The endoscope 100 may be in wired- or wireless-contact with personal viewer 120.

Personal viewer 120 is readily configured with a self-contained power supply 122 such as a battery, a receiver module 124 for receiving the wired or wireless signals, an image processor 126 for translating the received signals to one or multi-dimensional views in a head mounted display or heads up display (HUD) 128 or TV or video display monitor 130. Signals are readily sent via wired- or wireless-communication to a local area network (LAN) 132 such as by

Ethernet for internal communication systems. In addition signals from the endoscope 100 may be transmitted to an Internet modem and/or router 134 for computers and other data devices to access the patient images taken by the endoscope 100 anywhere in the world via wide area networks (WAN), WiFi, WiMAX, satellite, cellular telephone network, or other known or available wireless network connections.

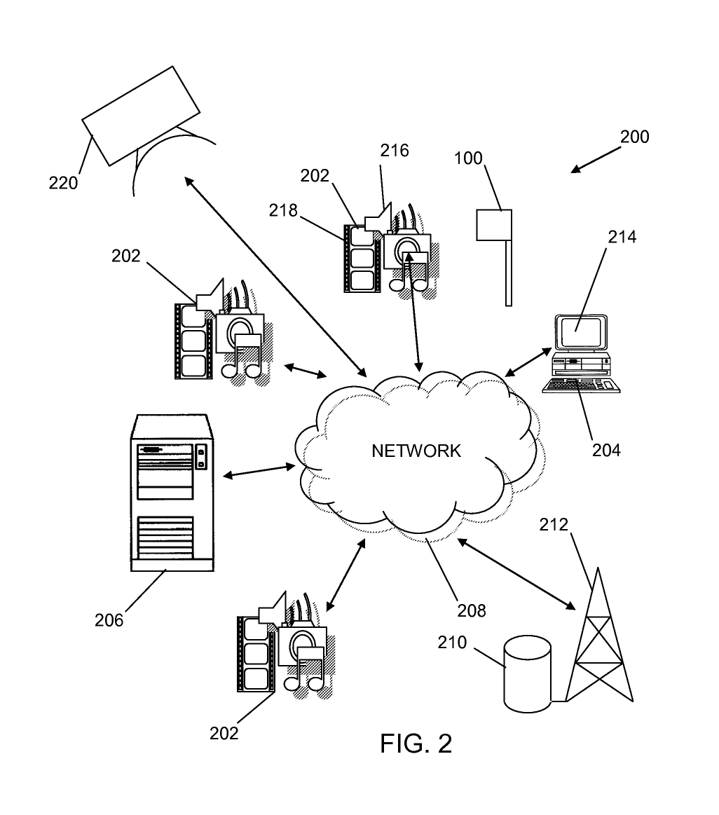

FIG. 2 is a schematic diagram illustrating an overall view of communication devices, computing devices, and mediums for interacting with the endoscope according to embodiments of the invention. The elements of the embodiments of the endoscope in FIG. 1 are included in the networks and devices of FIG. 2

The system 200 includes endoscope 100, the endoscope 100 including within an integrated enclosure optical tower function so as to eliminate the need of a separate optical tower, multimedia devices 202 and desktop computer devices 204 configured with display capabilities 214. The multimedia devices 202 illustratively include mobile communication and entertainment devices, such as cellular phones, mobile computing devices, tablet, TV, and personal displays that are wirelessly connected to a network 208. The multimedia devices 202 have video displays 218 and audio outputs 216. The multimedia devices 202 and desktop computer devices 204 are readily configured with internal storage, software, and a graphical user interface (GUI) for controlling and viewing images from the endoscope 100 according to embodiments of the invention. The network 208 is any type of known network including a fixed wire line network, cable and fiber optics, over the air broadcasts, satellite 220, local area network (LAN), wide area network (WAN), global network (e.g., Internet), intranet, etc. with data/Internet capabilities as represented by server 206. Communication aspects of the network are represented by cellular base station 210 and antenna 212. It is appreciated that the network 208 is in certain inventive embodiments a LAN and each remote device 202 and desktop device 204 executes a user interface application (e.g., Web browser) to contact the server system 206 and or endoscope 100 through the network 208. Alternatively, the remote devices 202 and 204 may be implemented using a device programmed primarily for accessing network 208 such as a remote client.

The software for viewing information from the endoscope 100 of embodiments of the invention, may be resident on the individual multimedia devices 202 and desktop computers 204, or stored within the server 206 or cellular base station 210. Server 206 may implement a cloud-based service for implementing embodiments of the endoscope with a multi-tenant database for storage of separate client data.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. An endoscope integrating functionality of an optical tower comprising:
an enclosure integrating the functionality of the optical tower, said enclosure containing said light source;
an elongated endoscope tube;
a camera;
an image processor, said camera in electrical communication with said image processor and supplies images and video to said image processor obtained via said elongated endoscope tube;
a light source, illuminating a viewing field of said endoscope via said elongated tube;
a transmitter module;
a communication interface;
a control interface; and
one or more of a power source, said one or more of said power source supplies powering said camera, said image processor, said light source, and said transmitter module.

2. The endoscope of claim 1 wherein said controls interface provides a user with the ability to control at least one of lighting intensity with said lighting module, said camera, parameters of imaging, communication parameters of said transmitter module and said communication interface.

3. The endoscope of claim 1 wherein the images are captured via a bundle of glass fibers that collect at a distal end of said elongated tube and provide the images to said camera and image processor at the proximal end of said elongated tube.

4. The endoscope of claim 1 wherein said transmitter module broadcasts the images and video obtained from said camera and image processor via predefined frequencies and protocols including at least one of WiFi (802.11 a,b,g,n), WiMax, or cellular.

5. The endoscope of claim 4 wherein said broadcasts are encrypted.

6. The endoscope of claim 1 wherein said communication interface routes the broadcast signals to an antenna.

7. The endoscope of claim 1 wherein said communication interface takes the image and videos and routes the image and videos to a cable interface for a wired connection.

8. The endoscope of claim 7 wherein the wired connection comprises at least one of composite video, s-video, universal serial bus (USB), high definition media interface (HDMI), digital video interface (DVI), or coax cable.

9. The endoscope of claim 1 wherein said transmitter module is configured for user selectable transmission frequencies and video channels for the endoscope to account for interfering signals.

10. The endoscope of claim 1 wherein said transmitter module is configured for the user to switch between two or more video channels to access additional informational content or operating room views.

11. The endoscope of claim 1 wherein said transmitter module is configured to overlay images onto a video feed from said camera is from another source or piece of medical equipment or monitoring device.

12. The endoscope of claim 11 wherein the overlay images represent at least one of patient vital sign information, augmented reality images, scans from computed tomography (CT), x-ray, or magnetic resonance imaging (MRI).

13. The endoscope of claim 1 wherein said power supply is directly connected to an AC electrical outlet.

14. The endoscope of claim 1 wherein said power supply is a battery.

15. The endoscope of claim 14 wherein said battery is rechargeable, said rechargeable battery is charged while connected to an outlet, or via a communication cable.

16. The endoscope of claim 1 wherein said endoscope is in wired or wireless contact with one or more personal viewers configured with a heads up display (HUD).

17. The endoscope of claim 1 wherein said endoscope is in wired or wireless contact with one or more presentation devices of a tablet, computer monitor, or a television.

18. The endoscope of claim 1 further comprising a conduit.

19. The endoscope of claim 1 wherein said one or more of said power source supplies act as a power source to peripheral devices, including, but not limited to, a vacuum device or other medical assist devices, via a USB connector or DC power receptacle built in to said enclosure.

20. The endoscope of claim 1 wherein said transmitter module is configured for a user to switch between two or more video channels to access shared views between two or more additional users operating additional endoscopes.
